(19) United States
(12) Patent Application Publication
(10) Pub. No.: US 2021/0060277 A1
(43) Pub. Date: Mar. 4, 2021

(54) Intubation Assistance Apparatus

(71) Applicants: John Alexander Cheyne, Vancouver (CA); Gillian Patricia Edworthy, Vancouver (CA)

(72) Inventors: John Alexander Cheyne, Vancouver (CA); Gillian Patricia Edworthy, Vancouver (CA)

(21) Appl. No.: 17/098,123

(22) Filed: Nov. 13, 2020

Related U.S. Application Data
(63) Continuation-in-part of application No. 16/252,199, filed on Jan. 18, 2019.

(51) Int. Cl. A61M 16/04

(52) U.S. Cl. CPC A61M 16/0463; A61M 2205/586; A61M 16/0418

(57) ABSTRACT

An intubation assistance apparatus having a connection body having a suction port, the intubation device port and a secondary suction port. The connection body has an internal passageway in fluid communication with the suction port, the intubation device port and the secondary suction port. The apparatus further having a rigid stylet in fluid communication with the intubation device port. The secondary suction port may have a thumb rest having a secondary suction opening being in fluid communication with internal passageway. The thumb rest has a concave surface to allow for ease of use by an operator.

### REFERENCE TO PENDING APPLICATIONS

This application claims the benefit of pending U.S. patent application Ser. No. 16/253,199, entitled Rigid Stylet Apparatus filed on Jan. 21, 2019.

### TECHNICAL FIELD

The present invention relates generally toward an apparatus for use with endotracheal intubation, and more specifically, an apparatus for use with an endotracheal tube with suction capability.

### BACKGROUND

Endotracheal intubation, usually referred to as intubation, is the placement of an endotracheal tube (ETT), which is typically a flexible plastic tube, into the trachea to maintain and secure an open airway and to serve as a conduit to administer oxygen and in occasion, certain medications. Intubation is normally facilitated by using a conventional laryngoscope to provide the operator with a view of the vocal cords to which the ETT will pass through.

The ETT is placed into the trachea through the use of an intubating stylet. The stylet is a metal wire with primary function to allow the user to bend the ETT to the optimal shape of the individual patient anatomy. The ETT shape should be maintained during intubation in its desired shape, as determined by the operator's manipulation of the stylet and therefore the ETT itself. There are multiple angles in which the ETT must go around, thus having a rigid stylet is critical in maneuvering the tube around the variable angles. An ETT without a stylet is flexible and can easily be caught up in any of these obstructive angles preventing a successful intubation. While there are different types of stylets, a rigid malleable stylet is the standard instrument used in intubation.

Viewing the vocal cords can be a critical challenge in ETT intubation. Increased secretions, vomit, and blood are the main fluid types preventing the view of the vocal cords. This fluid needs to be removed in order to view the vocal cords and pass the ETT through the vocal cords to secure the patients airway. ETT intubation generally requires a medically induced paralysis of the patient that prevents their cough reflex and swallowing capacity, resulting in the patient's inability to clear these secretions. There are multiple medical conditions that cause significant increased airway swelling (i.e. burns and toxidromes), blood in the airway or esophagus (gastrointestinal bleeds and pulmonary hemorrhages) or vomit/fecal matter in the airway. Secretions, blood or vomit can interfere with the operator's view of the vocal cords, resulting in a loss of oxygen to the patient. The longer that the foreign fluid remains in the airway, the greater the risk of harm to the patient. This fluid will inevitably enter the trachea and into the lungs resulting in hypoxia. Also, this fluid can lead to infection or chemical pneumonitis as the lungs fill with this fluid causing more inflammation in the lungs and a significant increase in morbidity and mortality. The Endotracheal tube (ETT) suction is therefore necessary to clear secretions and to maintain airway patency, and to optimize oxygenation and ventilation in a ventilated patient. ETT suction is typically conducted using a separate suction probe.

A challenge is the typical patient's airway can only accommodate two out of the three aforementioned devices needed to intubate due to the limited size of the airway, i.e. ETT, laryngoscope and suction probe, at any given time. As such, when secretions are in the airway of a patient being intubated, the endotracheal tube must be removed to make room for the suction probe as the laryngoscope always remains in the airway. When the suction probe is then removed, this allows the ETT to be reinserted for an additional attempt to secure the airway. During this extended time, the secretions may reoccur resulting in a repeat of the above. This can cause the patient to deoxygenate and decompensate. Further, any added hypoxic time can result in a significant increase in morbidity, even cardiorespiratory arrest and death.

The process of inserting the tube is called endotracheal intubation. This process typically includes an operator holding a laryngoscope (sometimes referred to a blade) in the one hand, i.e. the left hand, and is used to open the airway. The operator then inserts the ETT with the other hand, i.e. the right hand. Once the ETT is put into place, the laryngoscope is removed and the ETT is secured with tape and connected to ventilation equipment.

Sometimes, the insertion of the ETT can require assistance, such as when it is difficult to insert the ETT or where there is excess mucus or other secretion preventing an adequate insertion. Different apparatus can assist with the insertion of the ETT, such as a stylet or a bougie. While both a bougie and a stylet can be used to assist with intubation, they are two different pieces of equipment and require different techniques of operation.

A bougie is used as a guide to access the glottis by being inserted into the trachea through the glottis during direct laryngoscopy (DL). A bougie is a plastic linear, non-malleable (i.e. not able to hold a bent shape) airway device that typically measures approximately 3 feet in length. It is maneuvered into the airway alone and navigates the airway alone. The ETT is then “rail-roaded”, or moved, over this non-malleable flexible line into the vocal cords. The bougie has an inherent flexibility so that it can navigate the airway. As such due to the bougies' inherent flexibility, a bougie does not provide any structure, shape or stability to the ETT itself and functions solely as a conduit to which the ETT then slides over. More specifically, the bougie is not malleable and cannot change and hold a shape and therefor provides no shaping function to the ETT, and the ETT therefore also remains flexible. Further, due to the bougie's flexibility, once it has turned a corner in the airway, the operator has limited to no control over the tip of a bougie and can only advance it forward and backwards.

The typical technique for the utilization of a bougie includes, with the right hand placing the bougie within the trachea prior to the insertion of the ETT as it is used a “guidewire” over which the ETT can then be more easily advanced into the trachea. This is performed by placing the ETT over the free end of the bougie after it has been inserted into the trachea and then sliding the ETT along the bougie until the ETT is in place.

A stylet, in contrast, is used to provide the ETT with rigidity and shape molded to aid passage of the ETT into the trachea during insertion. A stylet is not a guide wire like a bougie. The stylet allows one to directly manipulate the ETT into the correct position. The stylet is placed inside the ETT prior to insertion. Due to the stylet being malleable, or able to hold a shape, it is typically bent toward the tip, or cuff, end of the ETT into a desired configuration, such as a “hockey stick” bend of no more than 35 degrees.

A stylet further allows the operator to directly maneuver the ETT within the airway. By bending the stylet tip anteriorly (and hence the ETT conforming to this desired design) and/or reverse loading an ETT on a stylet, the ETT tip can be manipulated at the level of the vocal cords in an anterior/posterior and left/right direction. More specifically, a stylet allows the ETT to be manipulated in all axis within the airway, an option that a bougie cannot perform. Once the ETT is in place, the stylet is removed from the ETT.

Because the stylet is located within the ETT prior to insertion, the process of inserting the ETT with the stylet is no different than the process without the stylet, namely an operator holding a laryngoscope in the left hand and is used to open the airway. The operator then inserts the ETT with the right hand. Once the ETT is put into place, the laryngoscope and stylet are removed. Unlike the process with the bougie, there is no sliding of the ETT over the stylet after insertion.

In some situations, suctioning of excess mucus or other secretions are needed during the insertion of the ETT. In the prior art, when suctioning is performed, the ETT is removed and a separate device, such as a Yankauer suction, is used. To date, suction has not been performed from within the ETT. Current practice demands that the ETT be removed to allow space for the suction device, suction then occurs, and the ETT is then reinserted. Often this is repeated multiple times as the fluid reaccumulates. This creates a time delay of the ventilation of the patient with a resulting increase in morbidity and mortality.

A stylet is housed within the ETT as its rigid nature could damage the airway if used directly within the airway. This intubation assist device would follow the same rules. The device's distal tip would not extend beyond the tip of the ETT such that the soft flexible portion of the ETT would always be the leading edge and prevent any direct contact of the stylet to the airway. The device, once suction is activated by the placement of a finger onto the secondary suction port, would generate suction through the distal portion of the ETT onto the stylet. The addition of a cap further increases suction, as the ETT itself becomes a vacuum system.

In the prior art, a bougie is typically not utilized in clinical practice to provide suction.

The flexible nature and narrow internal diameter of the bougie, along with the fact that it is inserted into the trachea prior the insertion of the ETT renders suction with a bougie clinically useless.

Further, when suction is utilized, suction needs to be specifically targeted, something not possible with a long flexible suction device. This is another reason why bougies are not utilized for providing suction.

U.S. Pat. No. 4,865,586 is a prior art attempt to resolve this challenge and is directed toward a suction guide for endotracheal intubation stylet. This device, however, provides continuous suction which results in fast deoxygenation. The device is a guide for the placement of an ETT rather than the stylet used for insertion, more specifically, this requires the ETT to be sleeved over the stylet after it has been passed through the cords. The estimated length of this patent for human use is longer than 3 feet.

U.S. Pat. No. 5,595,172 is another prior art attempt to resolve this challenge and is directed toward a suction stylet suited for use with an endotracheal tube. The two major differences are that this is not a rigid stylet, and the suction is not fully optional. This device is not designed with the use of a rigid stylet which is a critical part of the intubation process. Two design limitations of U.S. Pat. No. 5,595,172 results in oxygen continuously being removed from the patient's airway. The mechanics of the optional vent port are located a significant distance from the vacuum attachment. Also, the tube radius of the two lumens, specifically the tube leading to the vent port, relative to the other tube radius of the flexible plastic tube within the ETT are approximately 1:4. The result is a small amount of oxygen being continuously removed from the patient's airway even when the optional vent port is not being initiated.

Accordingly, there is a need for a device to address the above challenges.

### SUMMARY

The present invention relates generally toward an apparatus for use with endotracheal intubation, and more specifically, to an apparatus to assist with intubation having suction capability.

In one aspect, an intubation assistance apparatus is disclosed. This apparatus includes a connection body having a suction port and an intubation device port. The connection body has an internal passageway in fluid communication with the suction port and the intubation device port.

In some aspects, the suction port and the intubation device port are inline, while in other aspects, the suction port is substantially perpendicular to the intubation device port.

In some aspects, the intubation assistance apparatus further includes either a rigid stylet or an endotracheal tube that is in fluid communication with the intubation device port.

In some aspects, an intubation assistance apparatus is disclosed. The apparatus includes a connection body having a base, a suction port and an intubation device port. The connection body also has an end cap portion surrounding the intubation device port and extending away from the base. The connection body also has an internal passageway providing fluid communication between the suction port and the intubation device port.

In some aspects, the connection body further includes a secondary suction port wherein the internal passageway provides fluid communication between the suction port, the intubation device port, and secondary suction port.

In some aspects, the suction port and the intubation device port are inline, and the secondary suction port is substantially perpendicular to the suction port and the intubation device port. In other aspects, the secondary suction port and the intubation device port are inline, and the suction port is substantially perpendicular to the secondary suction port and the intubation device port.

In some aspects, the secondary suction port in fluid communication with the internal passageway and includes a thumb rest that has a concave surface to allow for ease of placement of the operator's thumb.

Other aspects and features of the present invention will become apparent to those ordinarily skilled in the art upon review of the following description of specific embodiments of the invention in conjunction with the accompanying figures.

### BRIEF DESCRIPTION OF THE DRAWING

In drawings which illustrate embodiments of the invention wherein similar characters of reference denote corresponding parts in each view.

FIG. 1 is a top perspective view of an embodiment of an apparatus of the present invention.

FIG. 2 is a bottom perspective view of an embodiment of an apparatus of the present invention.

FIG. 3 is a front view of an embodiment of an apparatus of the present invention.

FIG. 4 is a cross-sectional view of an embodiment of an apparatus of the present invention.

FIG. 5 is a cross-sectional view of an additional embodiment of an apparatus of the present invention.

FIG. 6 is a top perspective view of an additional embodiment of an apparatus of the present invention.

FIG. 7 is a bottom perspective view of an additional embodiment of an apparatus of the present invention.

FIG. 8 is a front view of an additional embodiment of an apparatus of the present invention.

FIG. 9 is a top view of an additional embodiment of an apparatus of the present invention.

FIG. 10 is a cross-sectional view of an additional embodiment of an apparatus of the present invention.

FIG. 11 is a cross-sectional view of an additional embodiment of an apparatus of the present invention.

FIG. 12 is a bottom perspective view of an additional embodiment of an apparatus of the present invention.

FIG. 13 is a front view of an additional embodiment of an apparatus of the present invention.

FIG. 14 is a cross-sectional view of an additional embodiment of an apparatus of the present invention.

FIG. 15 is a cross-sectional view of an additional embodiment of an apparatus of the present invention.

FIG. 16 is a cross-sectional view of an additional embodiment of an apparatus of the present invention.

FIG. 17 is a cross-sectional view of an additional embodiment of an apparatus of the present invention.

FIG. 18 is a cross-sectional view of an additional embodiment of an apparatus of the present invention.

### DETAILED DESCRIPTION OF THE INVENTION

The intubation assistance apparatus according to the present invention is a connection body that includes a suction port and an intubation device port, wherein the suction connection body is secured to an intubation device, such as an endotracheal tube (ETT) or rigid stylet. Suction functionality is provided through suction port.

Embodiments of the present invention are used to assist in the insertion of an endotracheal tube (ETT) to help a patient breathe. The ETT is a flexible plastic tube that is placed through the mouth into the trachea (windpipe). The endotracheal tube is then connected to a ventilator, which delivers oxygen to the lungs.

Embodiments of the present invention provide for a straightforward, safe apparatus that can provide suction during the insertion of an ETT. In one embodiment, the apparatus sits atop a rigid stylet, which then sits atop the ETT. In other embodiments, the apparatus sits directly atop the ETT, without the attached stylet. In some other embodiments, the capacity to allow the operator to provide suction on demand is provided. The operator may utilize this device with no additional assistance from another person while providing safe suction from the tip end of the ETT.

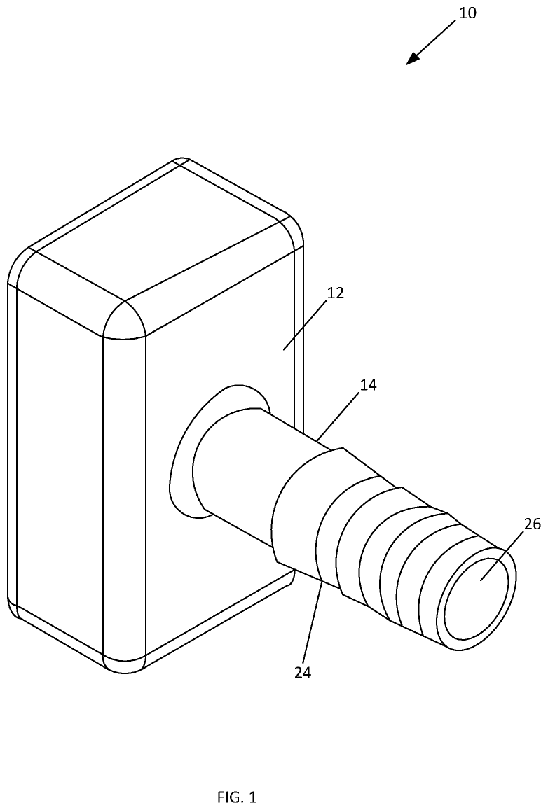

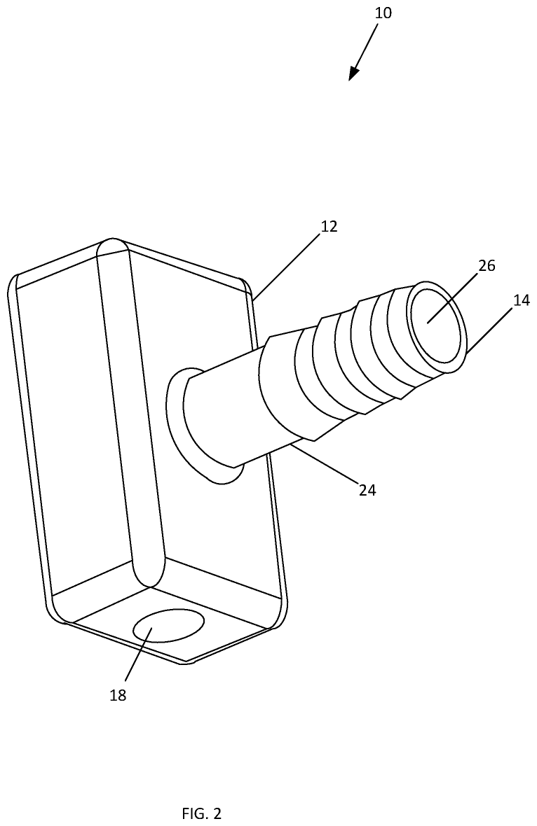

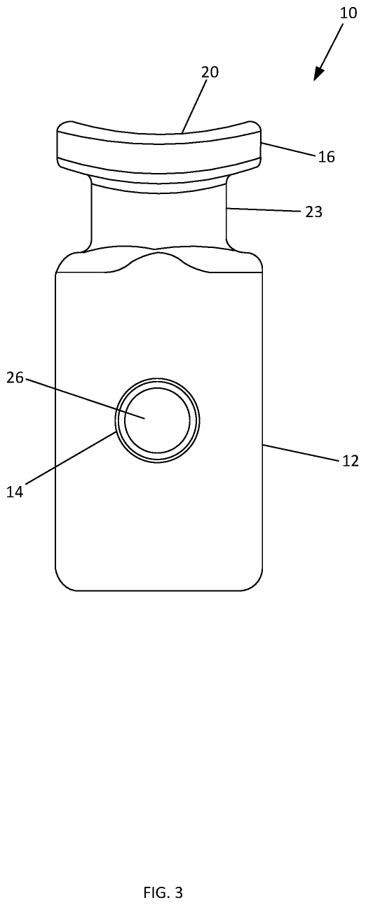

As illustrated in the FIGS. 1-4, an embodiment of an intubation assistance apparatus 10 of the present invention is disclosed. Apparatus 10 includes a connection body 12 having an intubation device port 18 and a suction port 14. Connection body 12 further includes an internal passageway 40 which is in fluid communication with suction port 14 and intubation device port 18.

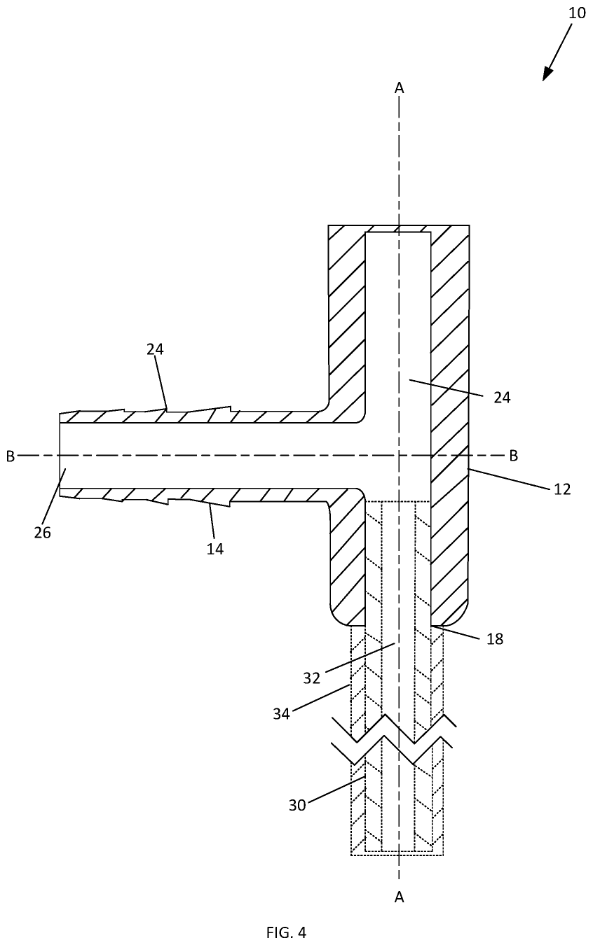

Intubation device port 18 is dimensioned to allow an intubation device 30 to be inserted therein, as illustrated in FIG. 4. Intubation device 30 is shown in dashed lines as it is not a required aspect of the present invention. It is included some, but not all, embodiments. Intubation device 30 may be any device that is configured to provide intubation, such as an ETT, or aid with the intubation, such as a rigid stylet. Intubation device 30 includes an intubation passageway 32 which allows for fluid communication with internal passageway 40. As apparatus 10 is designed for use with an intubation device 30, it is capable of handling and being utilized in all intubation situations which is not available in the prior art.

When intubation device 30 is a rigid stylet, it is designed to be placed inside an endotracheal tube (ETT) to assist with the insertion of the ETT into a patient's trachea. Suction port 14 is configured to provide suction through the internal passageway 40 to a patient's airway in order to remove harmful secretions.

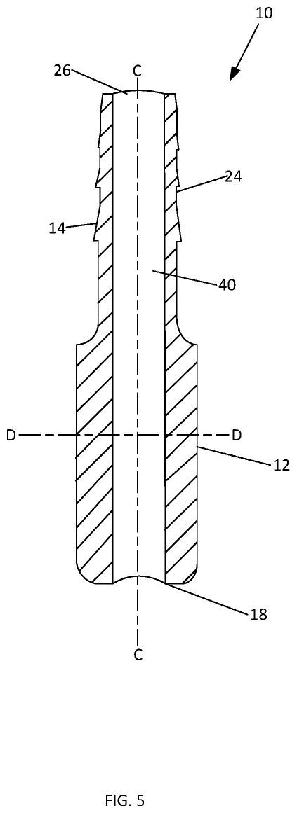

In this embodiment, intubation device port 18 is on a bottom surface of connection body 12, as shown by line AA. Suction port 14 is configured to be approximately perpendicular to intubation device port 18, as shown by line BB. This configuration allows for an ease and simplicity of use. Those skilled in the art, however, will recognize that other configurations of apparatus 10 may be utilized and are within the scope of the present invention. Other configurations, as illustrated in FIG. 5, may include suction port 14 and intubation device port 18 are in line with each other as shown by lines CC and DD.

In this embodiment, intubation device 30 is shown to be a ridge stylet with apparatus 10 sitting atop an ETT 34. This is illustrative and not meant to be limiting. Intubation device 30 may be an ETT without a rigid stylet.

In this embodiment, suction port 14 includes an elongated fitting 24 having an opening 26. Fitting 24 is designed to be secured to an external hose or other device but assist with the suctioning of the patient's throat during the intubation process. Fitting 24 may be a standard shape of fitting for tubing that connects to all wall vacuum systems to allow suction.

In operation, suction port 14 is connected to an external suction device which creates negative pressure within internal passageway 40 pulling airflow through rigid intubation passageway 32 and internal passageway 40 and exiting suction port 14. The allows for secretions and other items that could interfere with a patient's airflow, and operator's ability to intubate the patient, to be removed without the need for the removal of the ETT during the intubation process.

In other embodiments, the inventive apparatus may be utilized to deliver oxygen rather than creating a vacuum/suction. This can be accomplished by attaching an oxygen source to the vacuum port.

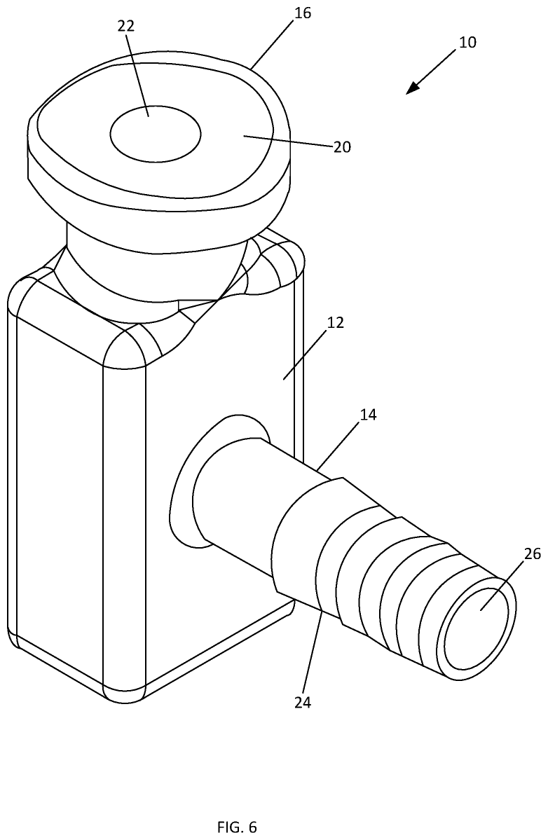

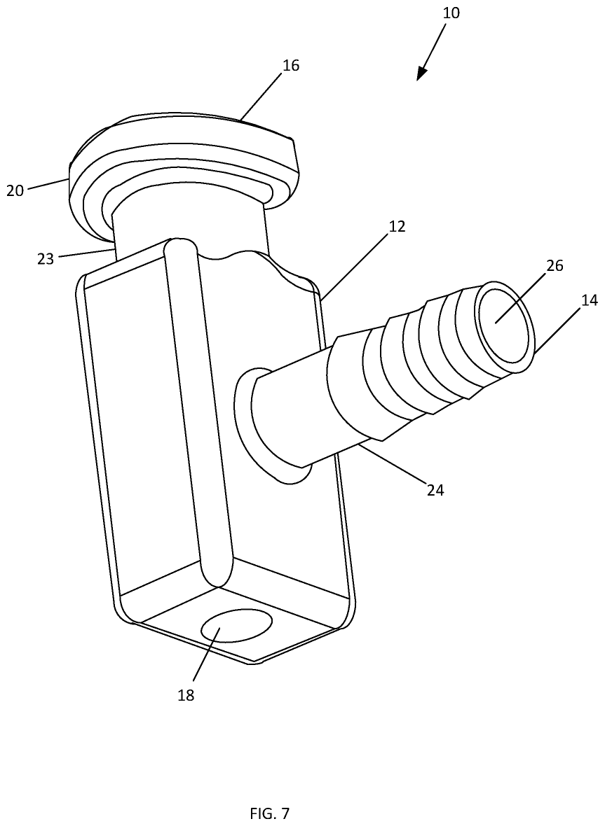

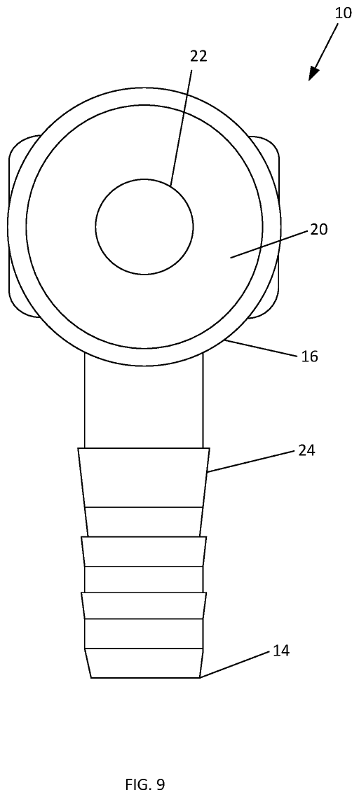

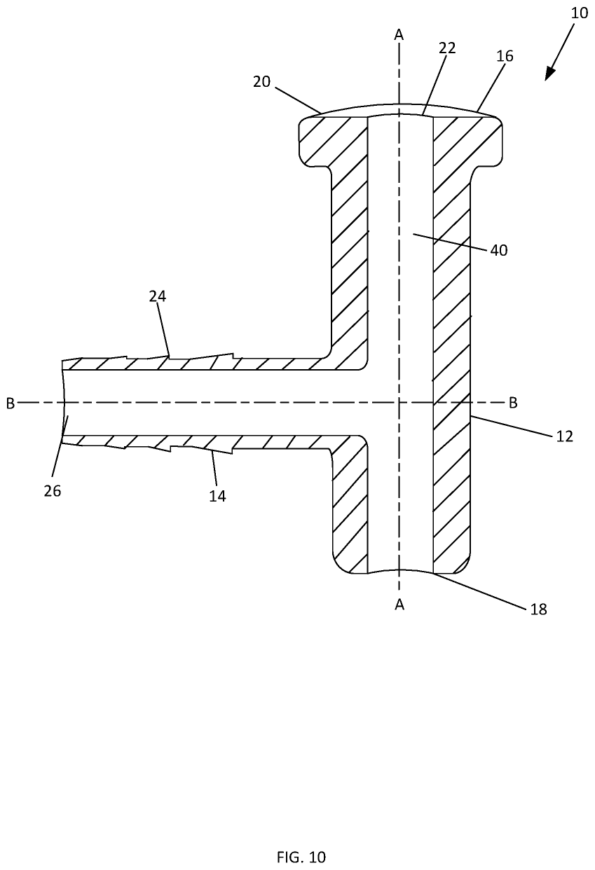

As illustrated in the FIGS. 6-10, an additional embodiment of an intubation assistance apparatus 10 of the present invention is disclosed. Apparatus 10 includes a connection body 12 having an intubation device port 18, a suction port 14 and a secondary suction port 16. Connection body 12 further includes an internal passageway 40 which is in fluid communication with suction port 14, intubation device port 18 and secondary suction port 16.

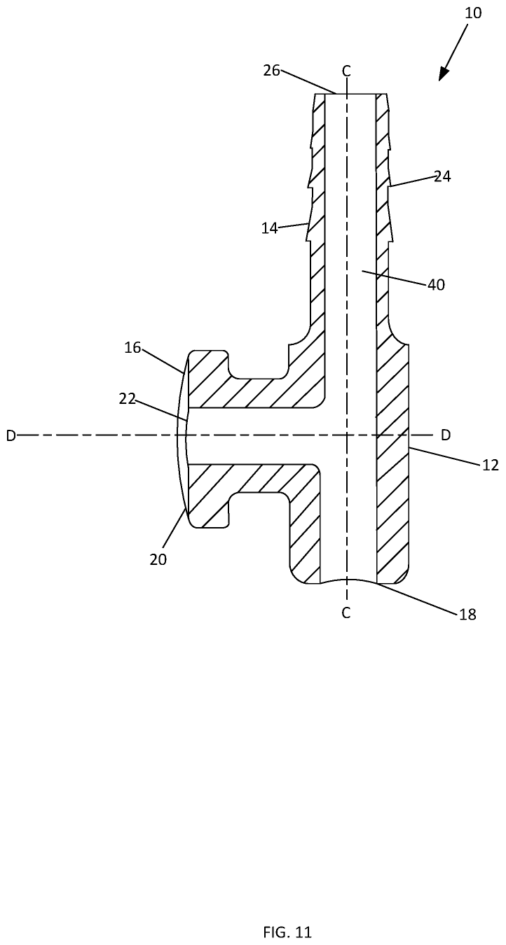

In this embodiment, secondary suction port 16 and intubation device port 18 are in line with each other, i.e. secondary suction port 16 is on a top surface of connection body 12 while intubation device port 18 is on a bottom surface of connection body 12, as shown by line AA. Suction port 14 is configured to be approximately perpendicular to secondary suction port 16 and intubation device port 18, i.e. suction port 14 extends from operator's thumb over the secondary suction port 16 while gripping connection body 12 during use, as shown by line BB. This configuration allows for an ease and simplicity of use. Those skilled in the art, however, will recognize that other configurations of apparatus 10 may be utilized and are within the scope of the present invention. Other configurations, as illustrated in FIG. 11, may include suction port 14 and intubation device port 18 are in line with each other while secondary suction port 16 is configured to be approximately perpendicular to secondary suction port 16 and intubation device port 18. Still other configurations may include suction port 14 to be oriented at any angle, i.e. 0-90 degrees, relative to secondary suction port 16 and/or intubation device port 18, as shown by lines CC and DD.

The use of the term secondary suction, the term pertains to the presence of a suction port with the option to block the port and initiate a vacuum. Further, when this feature is not present, i.e. no secondary suction port is present, a continuous suction actions occurring.

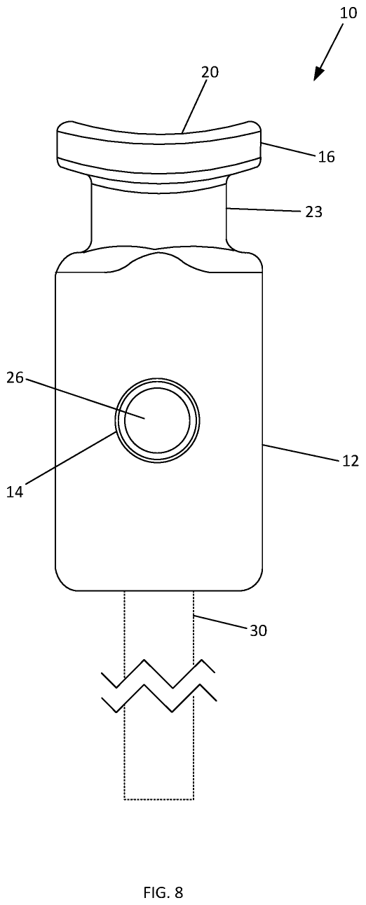

In this embodiment, secondary suction port 16 includes a thumb rest 20 having an secondary suction opening 22 which is in fluid communication with internal passageway 40. Thumb rest 20 is designed with a slight concave curvature as illustrated in FIG. 8. The slight concave curvature to allow for ease and accuracy of placement of the thumb during use. By having a slightly curved surface, the operator is more easily able to engage with secondary suction opening 22 during use as the operator's thumb may be guided to secondary suction opening 22 in the event of a slight misalignment of the operator's thumb during use.

Similar to previous embodiments, intubation device port 18 is dimensioned to allow an intubation device 30 to be inserted therein, as illustrated in FIG. 8. Intubation device 30 is shown in dashed lines as it is not a required aspect of the present invention. It is included some, but not all, embodiments. Intubation device 30 may be any device that is configured to provide intubation, such as an ETT, or aid with the intubation, such as a rigid stylet. Intubation device 30 includes an intubation passageway 32 which allows for fluid communication with internal passageway 40. As apparatus 10 is designed for use with an intubation device 30, it is capable of handling and being utilized in all intubation situations which is not available in the prior art.

In operation, suction port 14 is connected to an external suction device which upon the closure of secondary suction port 16 creates negative pressure within internal passageway 40 pulling airflow through rigid intubation passageway 32 and internal passageway 40 and exiting suction port 14. The allows for secretions and other items that could interfere with a patient's airflow, and operator's ability to intubate the patient, to be removed without the need for the removal of the ETT during the intubation process.

When secondary suction port 16 is not closed, i.e. when it is opened, air flow is pulled through secondary suction port 16 into internal passageway 40 and exiting suction port 14. This allows for intubation to occur without the need for suction.

By having the ability to optionally select when to engage suctioning through intubation device 30, the operate is given more control over the specific circumstances and conditions that are presented during intubation process. By having optional control. The capability of providing suction is readily available, while not providing constant suction which can lead to deoxygenation.

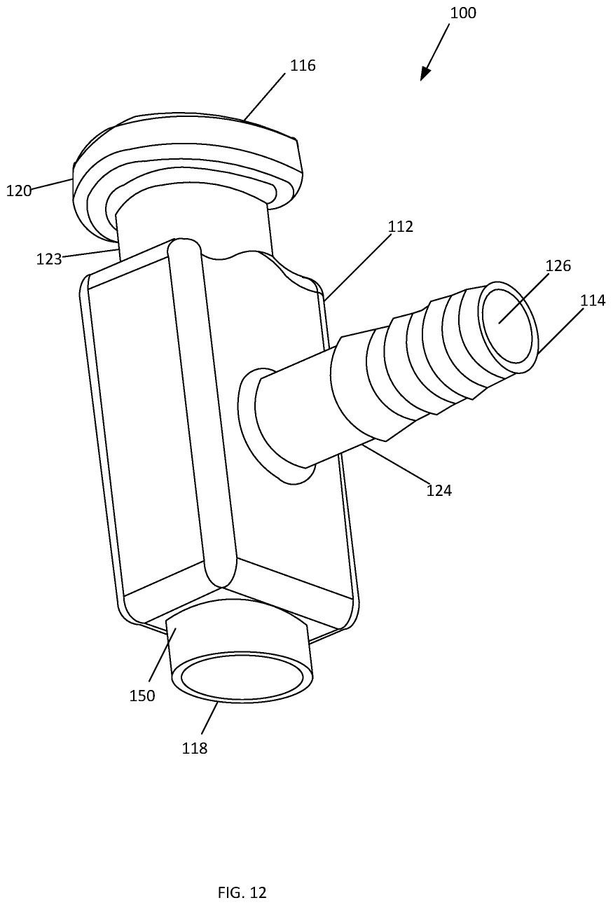

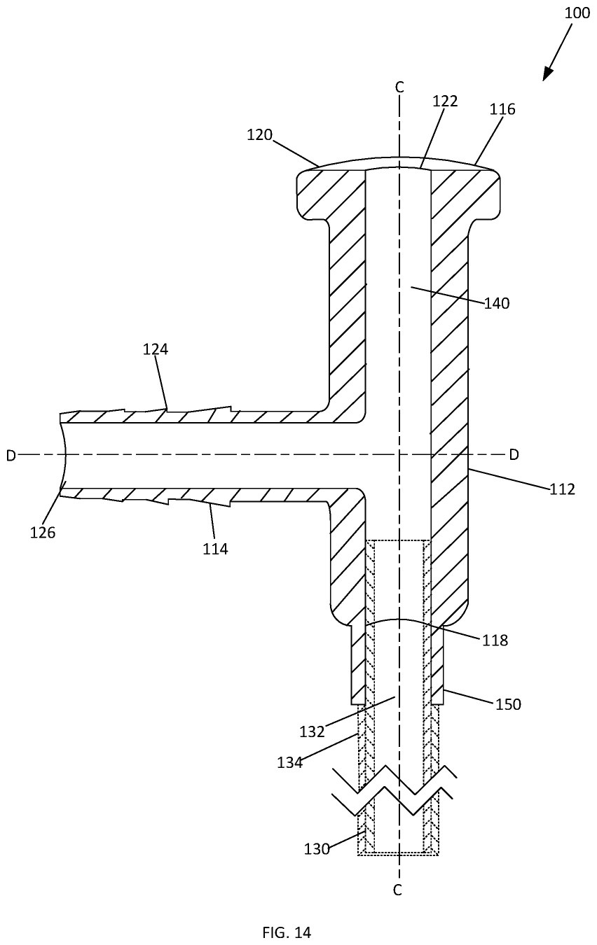

As illustrated in the FIGS. 12-14, an embodiment of an intubation assistance apparatus 100 of the present invention is disclosed. Apparatus 100 includes a connection body 112 having an intubation device port 118, a suction port 114 and a secondary suction port 116. Connection body 112 further includes an internal passageway 140 which is in fluid communication with suction port 114, intubation device port 118 and secondary suction port 116. Connection body 112 further includes an end cap portion 150 that is adapted to be mounted upon an end of an intubation device 130, such as an ETT. The apparatus 100 may or may not include an attached stylet, either embodiment can sit on top of an ETT.

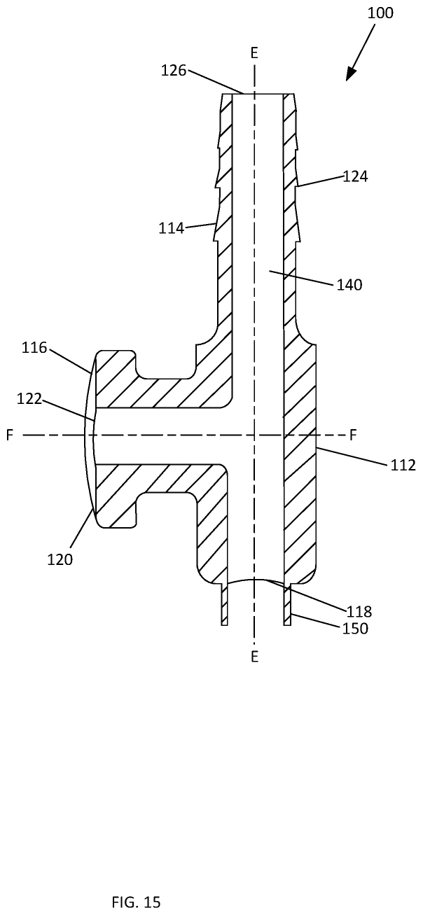

In this embodiment, secondary suction port 116 and intubation device port 118 are in line with each other, i.e. secondary suction port 116 is on a top surface of connection body 112 while intubation device port 118 is on a bottom surface of connection body 112, as shown by line CC. Suction port 114 is configured to be approximately perpendicular to secondary suction port 116 and intubation device port 118, i.e. suction port 114 extends from operator's thumb over the secondary suction port 116 while gripping connection body 112 during use, as shown by line DD. Those skilled in the art, however, will recognize that other configurations of apparatus 100 may be utilized and are within the scope of the present invention. Other configurations may include suction port 114 and intubation device port 118 are in line with each other while secondary suction port 116 is configured to be approximately perpendicular to secondary suction port 116 and intubation device port 118, as shown by lines EE and FF in FIG. 15. Still other configurations may include suction port 114 to be oriented at any angle, i.e. 0-90 degrees, relative to secondary suction port 116 and/or intubation device port 118.

In this embodiment, suction port 114 includes an elongated fitting 124 having an opening 126. Fitting 124 is designed to be secured to an external hose or other device but assist with the suctioning of the patient's throat during the intubation process. Fitting 124 may be a standard shape of fitting for tubing that connects to all wall vacuum systems to allow suction.

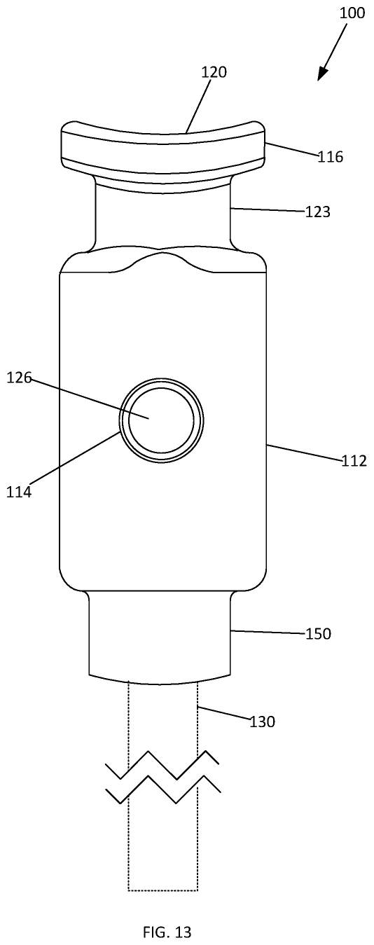

In this embodiment, secondary suction port 116 includes a thumb rest 120 having an secondary suction opening 122 which is in fluid communication with internal passageway 140. Thumb rest 120 is designed with a slight concave curvature as illustrated in FIG. 13. The slight concave curvature to allow for ease and accuracy of placement of the thumb during use. By having a slightly curved surface, the operator is more easily able to engage with secondary suction opening 122 during use as the operator's thumb may be guided to secondary suction opening 122 in the event of a slight misalignment of the operator's thumb during use.

In this embodiment, connection body 112 further includes an end cap portion 150 located at the base, or bottom side, of connection body 112. End cap portion 150 surrounds and encapsulates intubation device port 118. End cap portion 150 is configured to assist and improve the attachment of apparatus 100 to an ETT. It is adapted to sit on, or otherwise be affixed, to the top of intubation device 130 to provide a secure attachment and also a secure and powerful vacuum/suction system from within the intubation device 130. End cap portion 150 will allow the connection body 112 to directly attach to the top of the intubation device 130. The addition of end cap portion 150 can improve the suction generated as both intubation device 130 and connection body 112 can act as a vacuum/suction system when initiated by the operator.

Similar to previous embodiments, intubation device port 118 is dimensioned to allow an intubation device 130 to be inserted therein, as illustrated in FIG. 13. Intubation device 130 is shown in dashed lines as it is not a required aspect of the present invention. It is included some, but not all, embodiments. Intubation device 130 may be any device that is configured to provide intubation, such as an ETT, or aid with the intubation, such as a rigid stylet. Intubation device 30 includes an intubation passageway 132 which allows for fluid communication with internal passageway 140. As apparatus 100 is designed for use with an intubation device 130, it is capable of handling and being utilized in all intubation situations which is not available in the prior art.

In this embodiment, intubation device 130 is shown to be a ridge stylet with apparatus 100 sitting atop an ETT 34. This is illustrative and not meant to be limiting. Intubation device 130 may be an ETT without a rigid stylet.

In operation, suction port 114 is connected to an external suction device which upon the closure of secondary suction port 116 creates negative pressure within internal passageway 140 pulling airflow through rigid intubation passageway 132 and internal passageway 140 and exiting suction port 114. The allows for secretions and other items that could interfere with a patient's airflow, and operator's ability to intubate the patient, to be removed without the need for the removal of the ETT during the intubation process.

When secondary suction port 116 is not closed, i.e. when it is opened, air flow is pulled through secondary suction port 116 into internal passageway 140 and exiting suction port 114. This allows for intubation to occur without the need for suction.

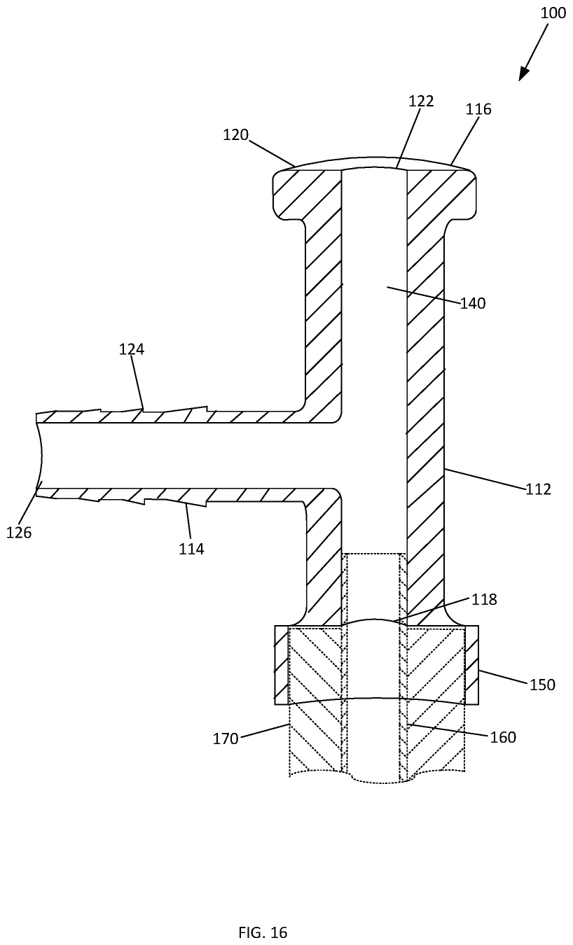

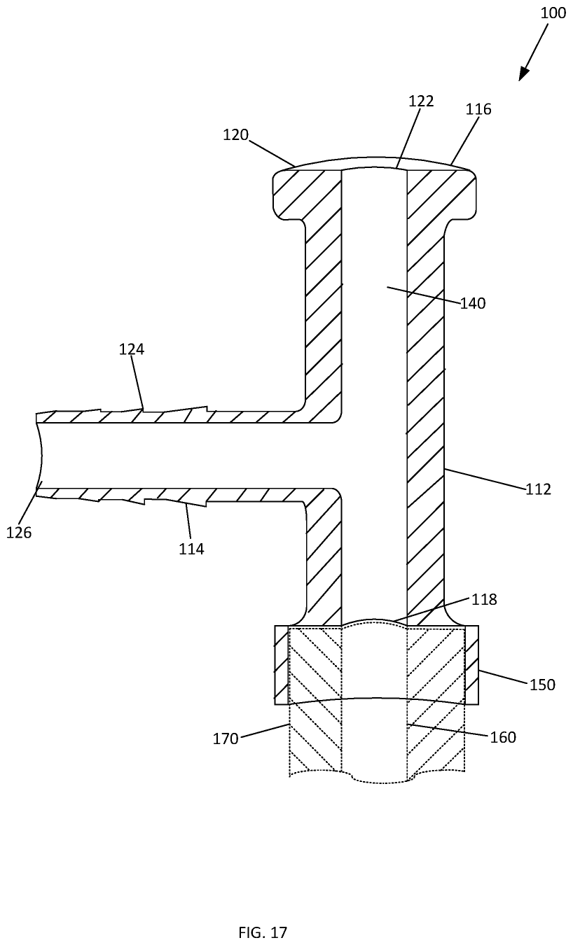

In some embodiments, as illustrated in FIG. 16, end cap portion 150 is dimensioned to accommodate an intubation device 160 that has a relatively wide diameter in comparison to the dimension of the connection body 112. FIG. 16 illustrates an embodiment where intubation device 160 is a stylet with a relatively wide diameter ETT 170. FIG. 17 illustrates an embodiment where ETT 170 is acting as the intubation device 160.

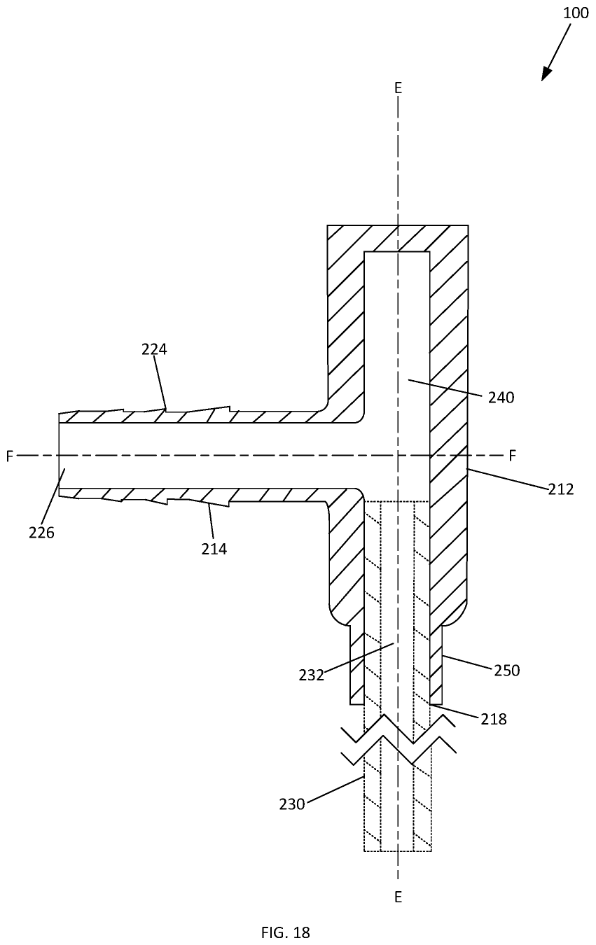

As illustrated in FIG. 18, an embodiment of an intubation assistance apparatus 200 of the present invention is disclosed. Apparatus 200 includes a connection body 212 having an intubation device port 218 and a suction port 214. Connection body 212 further includes an internal passageway 240 which is in fluid communication with suction port 214 and intubation device port 218.

In this embodiment, suction port 214 includes an elongated fitting 224 having an opening 226. Fitting 224 is designed to be secured to an external hose or other device but assist with the suctioning of the patient's throat during the intubation process. Fitting 224 may be a standard shape of fitting for tubing that connects to all wall vacuum systems to allow suction.

In this embodiment, connection body 212 further includes an end cap portion 250 located at the base, or bottom side, of connection body 212. End cap portion 250 surrounds and encapsulates intubation device port 218. End cap portion 250 is adapted to sit on, or otherwise be affixed, to the top of intubation device 230 to provide a secure attachment and also a secure and powerful vacuum/suction system from within the intubation device 130. End cap portion 250 will allow the connection body 212 to directly attach to the top of the intubation device 230.

Similar to previous embodiments, intubation device port 218 is dimensioned to allow an intubation device 230 to be inserted therein. Intubation device 230 is shown in dashed lines as it is not a required aspect of the present invention. It is included some, but not all, embodiments. Intubation device 230 may be any device that is configured to provide intubation, such as an ETT, or aid with the intubation, such as a rigid stylet. Intubation device 230 includes an intubation passageway 232 which allows for fluid communication with internal passageway 240. As apparatus 200 is designed for use with an intubation device 230, it is capable of handling and being utilized in all intubation situations which is not available in the prior art.

In operation, suction port 214 is connected to an external suction device creating negative pressure within internal passageway 240 pulling airflow through rigid intubation passageway 232 and internal passageway 240 and exiting suction port 214. The allows for secretions and other items that could interfere with a patient's airflow, and operator's ability to intubate the patient, to be removed without the need for the removal of the ETT during the intubation process.

While preferred embodiments of the present inventive concept have been shown and disclosed herein, it will be obvious to those persons skilled in the art that such embodiments are presented by way of example only, and not as a limitation to the scope of the inventive concept. Variations, changes, and substitutions may occur or be suggested to those skilled in the art without departing from the intent, scope, and totality of this inventive concept. Such variations, changes, and substitutions may involve other features which are already known per se and which may be used instead of, in combination with, or in addition to features already disclosed herein. Accordingly, it is intended that this inventive concept be inclusive of such variations, changes, and substitutions, and by no means limited by the scope of the claims presented herein.

I claim:

1. An intubation assistance apparatus comprising:
a connection body having a base, a suction port, an intubation device port, the connection body having an end cap portion surrounding the intubation device port and extending away from the base, the connection body having an internal passageway providing fluid communication between the suction port and the intubation device port.

2. The intubation assistance apparatus of claim 1, wherein the suction port and the intubation device port are inline.

3. The intubation assistance apparatus of claim 1, wherein the suction port is substantially perpendicular to the intubation device port.

4. The intubation assistance apparatus of claim 1 further comprising a rigid stylet in fluid communication with the intubation device port.

5. The intubation assistance apparatus of claim 1 further comprising an endotracheal tube in fluid communication with the intubation device port.

6. The intubation assistance apparatus of claim 1, wherein the connection body further comprises a secondary suction port and wherein the internal passageway providing fluid communication between the suction port, the intubation device port, and secondary suction port.

7. The intubation assistance apparatus of claim 6, wherein the suction port and the intubation device port are inline, and wherein the secondary suction port is substantially perpendicular to the suction port and the intubation device port.

8. The intubation assistance apparatus of claim 6, wherein the secondary suction port and the intubation device port are inline, and wherein the suction port is substantially perpendicular to the secondary suction port and the intubation device port.

9. The intubation assistance apparatus of claim 6, wherein the secondary suction port comprises:
a thumb rest having a secondary suction opening, the thumb rest having a concave surface, the secondary suction opening being in fluid communication with internal passageway.

10. The intubation assistance apparatus of claim 6 further comprising a rigid stylet in fluid communication with the intubation device port.

11. The intubation assistance apparatus of claim 6 further comprising an endotracheal tube in fluid communication with the intubation device port.
